(19) United States
(12) Patent Application Publication
(10) Pub. No.: US 2013/0131432 A1
(43) Pub. Date: May 23, 2013

(54) Selective Destruction of Cancer Cells via Tuned Ultrasound Resonance

(71) Applicant: Eric Kline, Rochester, MN (US)

(72) Inventor: Eric Kline, Rochester, MN (US)

(73) Assignee: INTERNATIONAL BUSINESS MACHINES CORPORATION, Armonk, NY (US)

(21) Appl. No.: 13/302,552

(22) Filed: Nov. 22, 2011

(51) Int. Cl. A61N 7/00; A61N 5/00

(52) U.S. Cl. 600/2; 601/2

(57) ABSTRACT

Disclosed is a method to use tuned resonant frequency ultrasonic energy for treatment of cancer that selectively destroys neoplastic cells while leaving surrounding healthy tissue minimally affected.

### BACKGROUND OF THE DISCLOSURE

Cancer is a severe medical problem. Many modalities of cancer treatment are available; however, many treatment regimes are difficult for the patient to endure, and many cancers remain incurable. Treatments that are more effective and more tolerable to the patient would be of benefit.

Cancerous/neoplastic cells can be larger or smaller than surrounding non-neoplastic cells depending on the type of cancer and the type of surrounding tissue (Tullberg, K F, and Burger, M M, Invasion Metastasis 5:1-15 (1985); Layfield, L J, and Ostrzega, N., Acta Cytol. 33:606-12 (1989)). Cancer cells typically have a larger and more disorganized nucleus than normal cells, due to an overproduction of DNA, and exhibit an increased frequency of mitosis. Thus, neoplastic cells can be distinguished from non-neoplastic cells based on differences in cell size, cell shape, nuclear size, nuclear shape, membrane morphology, and/or mitotic rate.

### BRIEF SUMMARY OF THE DISCLOSURE

Disclosed is a method to use tuned resonant frequency ultrasonic energy for treatment of cancer that selectively destroys neoplastic or cancer cells while leaving surrounding healthy tissue minimally affected. Specifically, the invention is focused on destroying cancerous tumors and growths at the cellular level, and further in leveraging the mitotic related distinctions and susceptibilities between normal and neoplastic cells.

### BRIEF DESCRIPTION OF THE FIGURES

FIG. 1. Size and volume comparison between normal and neoplastic cells.

FIG. 2. Estimated acoustic resonance frequency for biological structures of various lengths and modes.

FIG. 3. Resonance frequencies of various biologic structures.

### DETAILED DESCRIPTION OF THE DISCLOSURE

Tuned resonant frequency ultrasonic energy has been found to selectively destroy neoplastic or cancer cells while leaving surrounding healthy tissue minimally affected.

As used herein, a “neoplastic” or “cancer” cell means an abnormal cell exhibiting uncontrolled proliferation and potential to invade surrounding tissues. Neoplastic and cancer cells are characterized in part by increased rates of cell division or mitosis. This increase in mitotic rates provides one basis for discrimination between cancerous cells, which may (in certain lifecycle phases and/or under certain biologic conditions) reproduce frequently, and normal cells in the body, which tend to reproduce at a slower rate, if at all. Thus, treatment regimes designed to interrupt or interfere with mitotic cells can selectively target cancer cells to a significant degree of specificity and discrimination. This is the basis for many cancer treatments, such as chemotherapy and radiation therapy, both of which damage rapidly dividing cells. Unfortunately, such treatments also affect healthy tissue, which leads to significant side effects in the patient population.

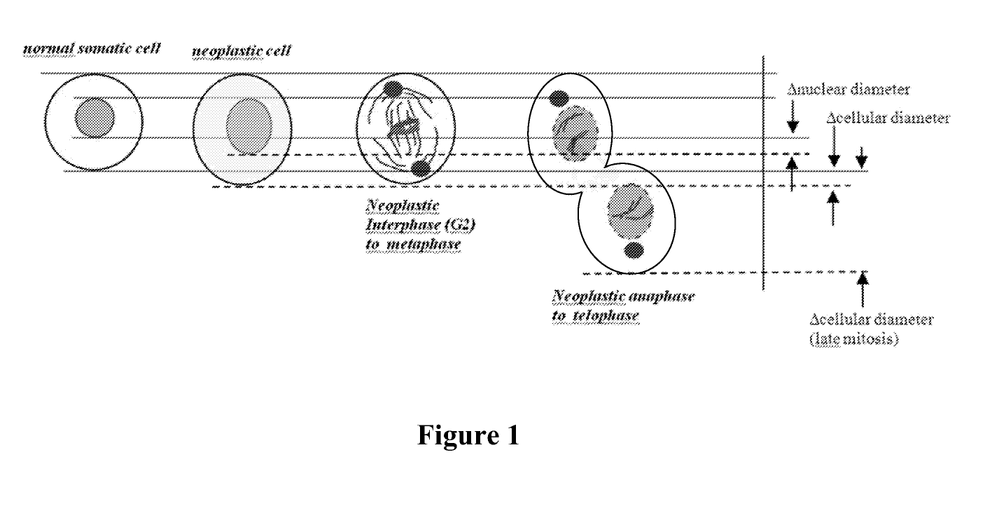

The progression of a cell from a non-neoplastic to a neoplastic state involves changes in many cellular aspects and processes resulting from multiple genetic mutations, including changes in cell size and/or shape, membrane composition, and mitotic rates. Cells undergoing neoplastic transition may grow larger or smaller than normal cells of that type (FIG. 1). Membrane composition can change, in part due to changes in cytoskeletal proteins, changes in lipid profiles, and alterations in expression of receptor proteins, signaling proteins, and other types of membrane proteins. Nuclear membranes of neoplastic cells also show significant changes. Cancer cells typically have large and disorganized nuclei, have multiple mutations in DNA, and undergo frequent mitosis. These differences between neoplastic cells and non-neoplastic cells can be used to distinguish and selectively target cancer cells.

TABLE 1
Comparison of Size between Normal and Neoplastic Cells

| | | Neoplastic Cell | Neoplastic Cell |
|---|---|---|---|
| Size Metric | Normal | (Interphase-G1 to | (Anaphase & |
| (major axis) | Somatic Cell | Metaphase) | Telophase) |
| Cell Diameter | 1.0 | 1.1 to 1.8 | 1.2 to 3.0 |
| Nuclear Diameter | 1.0 | 1.1 to 2.0 | 1.2 to 4.0 |
| Other Intercellular | 1.0 | 1.0 | 1.0 |
| Structural Sizes | | | |

Table 1 illustrates the viability of leveraging mitotic states by virtue of size or volume differences to differentiate between normal and neoplastic cells. Compared with the diameter of a normal somatic cell (normalized at 1), neoplastic cells have larger cellular and nuclear average diameters, particularly during the anaphase-telophase periods of cell division. These diametric differences can be used to selectively target mitotic neoplastic cells. In contrast to differences in cellular and nuclear diameters, the size of other intracellular structures, such as spindles, are nearly equivalent between normal and neoplastic cells. Thus, in order to employ differences in cellular and nuclear size as the basis of therapeutic treatment, neoplastic cells in mitotic stages are preferentially targeted.

Ultrasound energy has been previously studied as a means for selective destruction of target cells within the body, and as a means to augment other forms of cancer therapy; however, cancer treatment with ultrasound under existing protocols is not generally practiced. The use of ultrasonic energy to treat cancerous conditions has significant potential, but present treatment regimes are not efficacious enough to produce highly therapeutic results in patients.

Herein is disclosed an improved method for targeted tumor cell destruction based on tuned resonant frequency ultrasonic energy. The methods of the invention show superior ability to damage cancer cells while minimizing damage to surrounding tissues.

Tuned resonant frequency ultrasound (“TRFU”) refers to acoustic/ultrasound energy that is tuned to resonate at a frequency corresponding to the size of the cellular structure targeted for disruption. This tuned ultrasound energy can damage or destroy cells by mechanisms such as:
a) cavitation, wherein the collapse of the cavity creates a strong local mechanical shock wave, and/or
b) mechanical damage or breakage of intra-cellular structures due to excitement of various vibrational, torsional, or translational modes within the resonating cellular or intracellular structure.

These mechanisms disrupt and break cellular components such as cellular/nuclear membranes, chromosomes/DNA, and/or other cellular structures or cellular proteins.

In one embodiment, TRFU employs ultrasonic energy in a frequency fundamental range of about 1 MHz to 1,500 MHz, with the range about the fundamental being variable, such as +/−5% to +/−15% of the calculated frequency fundamental. A clinical treatment regime can include ultrasonics tuned to the one half to the third harmonic of the fundamental, based on the cellular or intracellular structural target and the desired vibrational, torsional or translational mode to be excited.

In a second embodiment, the bandwidth of the ultrasound energy is discrete nominal harmonics of the frequency fundamental. In a further embodiment, the passband of the ultrasonic energy is either 0.5, 1.0, 2.0 or 3.0 times the frequency fundamental (+/−5% to 15%); the passband can alternately or additionally include other fixed harmonics of the fundamental (ie, 1.5, 2.5, 3.5, etc.). The term “about”, as applied throughout this disclosure, is defined as falling within +/−5% to +/−15% of the suggested level.

TRFU is preferred with power levels that specifically affect the target material with little or no detrimental effects on surrounding, nonresonant structures or surrounding structures having different specific resonance frequencies. Power intensity is dependent on factors including the adjacent or surrounding tissue, target material, and location and depth of the target cells. Power, power intensity (horn/transducer size) and exposure duration is determined based on, i.e., the type of cancer, size of the tumor, location of the tumor within the body, and other collateral factors such as the type of normal tissue surrounding the tumor. For TRFU, the power intensity can be from about 0.01 to about 1×10¹¹ W/m². The power intensity can preferably be approximately 10 to 100,000 W/m². The power intensity is most preferably in the range of about 100 to about 10,000 W/m².

TRFU can target changes in cancer cell size. For example, neoplastic cells that are larger or smaller than adjacent non-neoplastic cells can be selectively targeted by tuning the frequency to resonate with the diameter or major axis dimension of the neoplastic cells. This would cause maximum damage to the cells of the target size, while causing less damage to cells falling outside of the nominal neoplastic diameter or major axis dimensional range. Neoplastic cell diameters or major axis dimensions, particularly in neoplastic cells that are larger than adjacent non-neoplastic cells, are at maximum susceptibility to TRFU between the mitotic stages of anaphase to telophase, due to the larger cellular dimensions at those stages that would increase the size differentiation between normal and abnormal cells.

To target a cancer cell for damage or destruction, the frequency range for TRFU is 0.5 to 3.0 times the average maximum dimension of a neoplastic cell between the anaphase to telophase stages of mitosis. For example, for a neoplastic cell with an anaphase to telophase diameter or major axis dimension of approximately 60 microns, the frequency range would be 12.5 MHz to 75 MHz to resonate across the cellular diameter. For cancer cells of other average diameters, the frequency would be tuned to resonate according to the average diameter of those cells based on a frequency range of 0.5 to 3.0 times the average maximum dimension.

Harmonic resonance, at power density settings of >0.2 W/cm², and selective to cellular or intra-cellular structure size, can damage neoplastic cells while leaving normal/somatic cells substantially unaffected, according to EQ 1:

$$f = n \frac{v}{λ} \qquad EQ 1$$

where f is frequency; n is the “mode” of the fundamental (ie, 0.5, 1.0, etc.), v is the acoustic wave propagation velocity in the affected tissue, and λ represents the length of the major axis of the biological structure (ie, cell or nuclear diameter, etc.) being irradiated.

Per EQ 1, assuming for simplicity sake that the average acoustic propagation velocity (v) in biological tissues is approximately 1500 m/s, the nominal ultrasound frequency for the one half to the third harmonic will range between ˜25 MHz to 150 MHz based on a neoplastic cell diameter of approximately 30 microns (i.e., interphase to metaphase). Ultrasonic generators are readily able to generate an energy spread spectrum in this range.

The frequency that matches a particular acoustic wavelength depends on the composition of the target material, according to the EQ 2:

v=f*λ EQ 2

where velocity (v) refers to the speed of the acoustic wave propagation (the speed of sound) in the medium of the target material. Although the speed of sound varies among various biological tissues, it is roughly equivalent to the speed of sound in water (1,500 m/s), since most living bodies or body samples mainly contain water (except lung and bone tissue). Using the speed of sound in water as the velocity of the acoustic wave, and using the target material size (ie, cell diameter, nucleus diameter, spindle length, etc.) as the rough equivalent of the wavelength, the approximate range of resonant frequencies in target materials and surrounding substances can be determined. Known speeds of sound in specific tissues of a living body include: liver at about 1550 n/s; muscle at about 1580 n/s; fat at about 1459 n/s; brain at about 1560 m/s; kidney at about 1560 m/s; spleen at about 1570 m/s; blood at about 1575 m/s; bone at about 4080 n/s; lung at about 650 m/s; lens of eye at about 1620 m/s; aqueous humor at about 1500 m/s; and vitreous humor at about 1520 m/s. Resonant frequency ranges for target materials comprised of tissues with acoustic velocities different from the speed of sound in water, are derived using the same equation (velocity/wavelength) and correlated to the charted ranges listed below, depending on the speed of sound in the target material and/or surrounding tissue.

TABLE 2
Estimated acoustic resonance frequencies for biologic structures
of various lengths (L) and modes (n).

| L | n | f | log(f) |
|---|---|---|---|
| 1.00E−06 | 0.5 | 7.50E+08 | 20.4356 |
| 1.00E−06 | 1.0 | 1.50E+09 | 21.1287 |
| 1.00E−06 | 2.0 | 3.00E+09 | 21.8219 |
| 1.00E−06 | 3.0 | 4.50E+09 | 22.2273 |
| 1.00E−05 | 0.5 | 7.50E+07 | 18.1330 |
| 1.00E−05 | 1.0 | 1.50E+08 | 18.8261 |
| 1.00E−05 | 2.0 | 3.00E+08 | 19.5193 |
| 1.00E−05 | 3.0 | 4.50E+08 | 19.9248 |
| 1.00E−04 | 0.5 | 7.50E+06 | 15.8304 |
| 1.00E−04 | 1.0 | 1.50E+07 | 16.5236 |
| 1.00E−04 | 2.0 | 3.00E+07 | 17.2167 |
| 1.00E−04 | 3.0 | 4.50E+07 | 17.6222 |

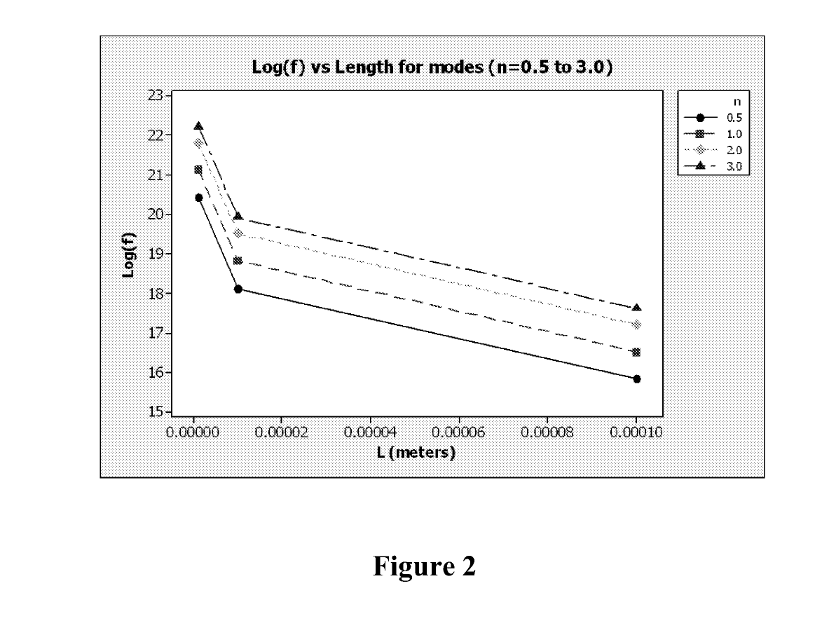

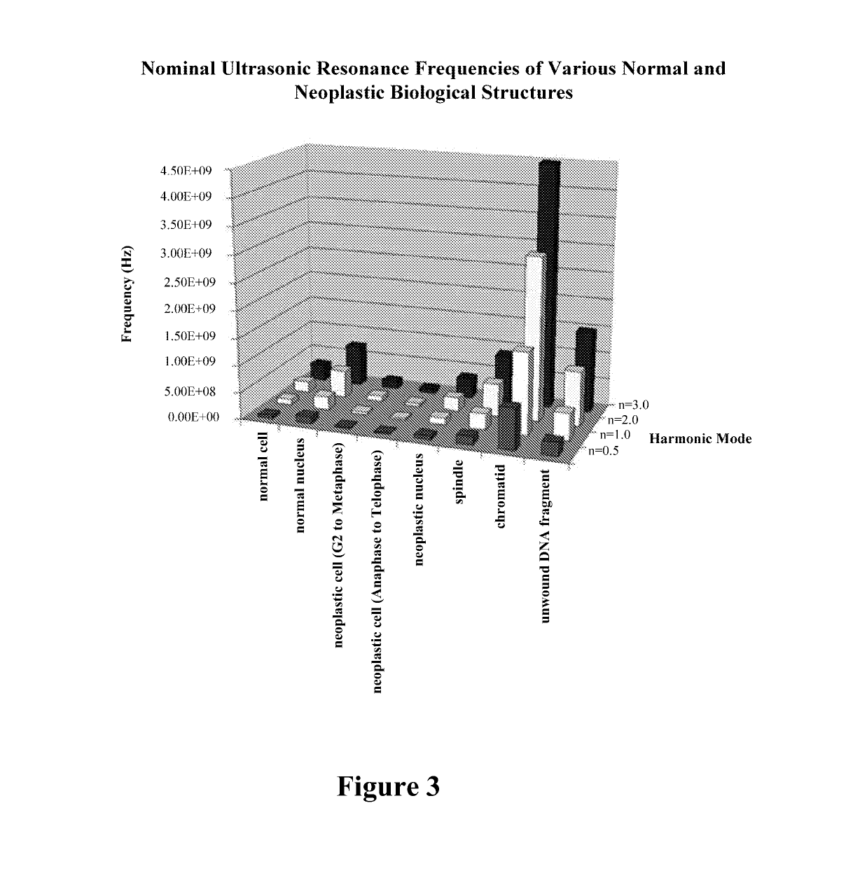

Table 2 and FIG. 2 illustrate the estimated resonance frequencies for biological structures of specific lengths, given modes of 0.5 to 3.0. As the length of the biological structure increases and/or mode decreases, the frequency decreases. Table 3 and FIG. 3 illustrate resonant frequencies for structures of given length for harmonic mode n=0.5, 1, 2, or 3.

TABLE 3
Example Calculated Frequencies for Various Target Biologic Structures

| Biologic | frequency for n = 0.5, 1, 2, or 3 | | | | |
|---|---|---|---|---|---|
| Structure | L | 0.5 | 1 | 2 | 3 |
| normal cell | 1.50E−05 | 5.00E+07 | 1.00E+08 | 2.00E+08 | 3.00E+08 |
| normal | 6.00E−06 | 1.25E+08 | 2.50E+08 | 5.00E+08 | 7.50E+08 |
| nucleus | | | | | |
| neoplastic | 3.00E−05 | 2.50E+07 | 5.00E+07 | 1.00E+08 | 1.50E+08 |
| cell (G2 to | | | | | |
| Metaphase) | | | | | |
| neoplastic | 6.00E−05 | 1.25E+07 | 2.50E+07 | 5.00E+07 | 7.50E+07 |
| cell | | | | | |
| (Anaphase to | | | | | |
| Telophase) | | | | | |
| neoplastic | 1.20E−05 | 6.25E+07 | 1.25E+08 | 2.50E+08 | 3.75E+08 |
| nucleus | | | | | |
| spindle | 5.00E−06 | 1.50E+08 | 3.00E+08 | 6.00E+08 | 9.00E+08 |
| chromatid | 1.00E−06 | 7.50E+08 | 1.50E+09 | 3.00E+09 | 4.50E+09 |
| unwound | 3.00E−06 | 2.50E+08 | 5.00E+08 | 1.00E+09 | 1.50E+09 |
| DNA | | | | | |
| fragment | | | | | |

TRFU can target the cellular or nuclear diameter of a cancer cell. To target a cancer cell nucleus for damage or destruction, the frequency range for TRFU is 0.5 to 3.0 times the average nuclear diameter of the neoplastic cell in the telophase stage of mitosis. For example, for a neoplastic cell with an average prophase nuclear diameter of approximately 12 microns, the frequency range would be 62.5 MHz to 375 MHz to resonate across the nuclear diameter.

Tuned resonant frequency can also damage or destroy neoplastic cells by fatally disrupting or disorganizing intracellular life-critical structures (such as mitochondria, metabolic structures, cellular proteins) and/or reproductive-critical structures, such as chromosomes, DNA, and mitotic spindles. During mitosis, chromosomes and unwinding DNA are highly susceptible to acoustic energy. Very large mechanical moments driven by the acoustic energy will produce shocks and torques all along these structures, causing multiple breaks in DNA that are beyond the capacity of endogenous DNA repair machinery to correct. Once DNA is broken, cell survival is diminished. Even if the cell does not perish immediately, the cell will not be able to replicate with damaged DNA so cellular proliferation is reduced.

TRFU can target mitotic cells between late prophase and telophase stages, due to the lack of nucleus and existence of spindles and DNA strands that are highly sensitive to ultrasound energy. TRFU can be tuned to the intracellular structure, such as the average spindle or DNA fragment length. For example, for an average spindle length of 5-10 microns, the frequency range would be 150 MHz to 900 MHz.

This disclosure provides a method for reducing or eliminating neoplastic cells in a subject comprising administering to said subject a therapeutically effective treatment regime of tuned resonant frequency acoustic energy, wherein said resonant frequency is within the range 1 MHz to 1,500 MHz, and wherein said treatment regime reduces or eliminates said neoplastic cells.

In one embodiment, the tuned resonant frequency ultrasound bandwidth is calculated to be 0.5 to 3.0 times the average cellular diameter of a mitotic neoplastic cell of said neoplastic cell population, such cell being in the anaphase to telophase stage of mitosis. In a specific embodiment, the tuned resonant frequency range is 12.5-75 MHz.

In another embodiment, the tuned resonant frequency ultrasound bandwidth is calculated to be 0.5 to 3.0 times the average nuclear diameter of a mitotic neoplastic cell of said neoplastic cell population, such cell being in the late prophase stage of mitosis. In a specific embodiment, the tuned resonant frequency range is 63-375 MHz.

In a further embodiment, the tuned resonant frequency ultrasound bandwidth is calculated to disrupt the average spindle length of a mitotic neoplastic cell of said neoplastic cell population, such cell being in the prophase to telophase stage of mitosis. In a specific embodiment, the tuned resonant frequency range is 150-900 MHz.

In another embodiment is provided a method for reducing or eliminating neoplastic cells in a subject comprising administering to said subject a therapeutically effective treatment regime of tuned resonant frequency ultrasound energy, wherein said regime consists of two or more different resonant frequencies tuned to resonate with cellular structures of mitotic cells, and wherein said treatment regime reduces or eliminates said neoplastic cells.

A specific embodiment provides a method for reducing or eliminating neoplastic cells in a subject comprising administering to said subject a therapeutically effective treatment regime of tuned resonant frequency ultrasound energy, wherein said regime comprises administration of two or more different resonant frequencies and wherein said two or more different frequencies fall within two or more of the following ranges: 0.5 to 3.0 times the average cellular diameter of a mitotic neoplastic cell of said neoplastic cell population, such cell being in the anaphase to telophase stage of mitosis; 0.5 to 3.0 times the average nuclear diameter of a mitotic neoplastic cell of said neoplastic cell population, such cell being in the late prophase stage of mitosis; and 0.5 to 3.0 times the average spindle or DNA fragment length of a mitotic neoplastic cell of said neoplastic cell population, such cell being in the metaphase to telophase stage of mitosis.

In a further specific embodiment is provided a method for reducing or eliminating neoplastic cells in a subject comprising administering to said subject a therapeutically effective treatment regime of tuned resonant frequency ultrasound energy, wherein said regime comprises administration of two or more different resonant frequencies and wherein the two or more different frequencies fall within two or more of the following ranges: 25-150 MHz; 150-900 MHz; and 250-1,500 MHz.

In an additional embodiment, the passband of the ultrasound energy is discrete between 0.5 to 3.0 times the frequency fundamental. In this embodiment, the passband of the ultrasonic energy is either 0.5, 1.0, 2.0 or 3.0 times the frequency fundamental (+/−5 to 15%); the passband can alternately or additionally include other fixed harmonics of the fundamental (ie, 1.5, 2.5, 3.5, etc.). For example, a neoplastic cell with an average diameter of 30 microns would resonate with the following alternative frequencies (within +/−15% of the nominal): 25 MHz, 50 MHz, 100 MHz, or 150 MHz.

In an additional embodiment, the passband or bandwidth of the ultrasonic energy is specifically selected in the frequency domain to disrupt biologic structures according to maximum selective destructive potential based on morphological or structural changes, such as the transformation of neoplastic cells into a more cylindrical or other shape, such as extension of pseudopod-like structures during metastasis (choosing resonance modes not only for size but also based on geometry type), or during particularly sensitive phases (ie, during telophase when the nuclear membranes are reforming). The passband or bandwidth can further be selected in order to utilize specific harmonic modes (ie, n=0.5) which exhibit particular vibratory, torsional or translational characteristics, which may be more deleterious for some structures than others. In this way biologic targeting can be even more selective and specific to neoplastic cells and cell structures.

A sample of neoplastic cells can be removed from a cancer patient by various methods, for example, biopsy (when the cancer is within solid tissue) or blood sample (when the cancer is found within the bloodstream). The sample of neoplastic cells can then be examined to identify cellular morphology, diameters of cellular and nuclear membranes, mitotic spindle size, etc. Once the neoplastic cell population has been examined and characterized, resonant frequency ranges can be calculated based on preferred targets for damage or destruction.

To apply the methods of the invention to a subject in need of treatment, the cancerous region or regions of the subject are exposed to the disclosed TRFU at the prescribed frequency, power level 10 to 100,000 W/m², and time for clinically efficacious results (i.e., 1 exposure per week for 10 weeks). Alternatively, the subject's whole body can be exposed in a whole body emitter such as an anechoic chamber. Even with resonant frequency ultrasound tuned to an optimal frequency for selective damage of cancer cells, there will be some damage to normal tissue; therefore, an effort to maximize neoplastic versus non-neoplastic discrimination is desired.

Treatment is continued until neoplastic cell proliferation has stopped and/or neoplastic cells are destroyed, or until damage to surrounding somatic tissue exceeds a certain prescribed level. Treatment with TRFU can optionally be combined with one or more art-recognized additional anticancer treatments, such as surgery, radiation and/or chemotherapeutic agents. TRFU, alone or in combination with anticancer treatments such as radiation and chemotherapeutic agents, can be particularly useful for treating inoperable tumors and in other specialized situations where less invasive methods of treatment are desired.

Equipment (ie, ultrasonic generators) for providing TRFU treatment is known in the art. Any suitable ultrasound device that can be programmed to generate acoustic waves with characteristics such as frequency, mode, pulse duration, shape, and repetition rate can be utilized to generate TRFU frequencies utilized herein. Ultrasound equipment manufacturers at the time of filing this application include Acuson, Philips Ultrasound, B-K Medical A/S, Esaote Biomedica, GE Ultrasound, Hewlett Packard, Krotz, Medison, Siemens, Shimadzu, Toshiba, Hitachi Medical Systems, Honda Electronics, SonoSite, Kontron Medical, Fukuda Denshi, and others.

### Example

Neoplastic cells are isolated from a patient in need of treatment for a cancerous condition. The isolated neoplastic cells are characterized in such terms as the average cellular and nuclear diameters and shapes, cellular morphology, and target intracellular structure (including mitotic spindle and DNA strand) size. Further information is acquired, including type of cancer, tumor size, and number and location of neoplastic cell masses in the body. Following characterization of the cancer and neoplastic cell sample population, treatment with tuned resonant frequency ultrasound is calculated. Treatment is calculated based on the type of cancer cell and mitotic rate, the susceptibility of the cell, nucleus or intracellular structures to ultrasonic damage and the resistance of normal healthy cells to damage at a given ultrasonic frequency, power level and exposure regime.

The frequency passband or bandwidth for TRFU is calculated for disruption based on cellular and nuclear diameters, and spindle size. First, the average diameter of a neoplastic cell from the patient sample is characterized at 30 microns. TRFU is calculated at 0.5 to 3.0 times 30 microns, providing a frequency range of 25 MHz to 150 MHz to resonate across the cellular diameter.

The frequency range for TRFU is further calculated for disruption based on nuclear diameter. The average maximum dimension of neoplastic nuclei from the patient sample in the early prophase stage of mitosis is characterized at 12 microns. TRFU is calculated at 0.5 to 3.0 times 12 microns, providing a frequency range of 63 MHz to 375 MHz to resonate across the nuclear diameter.

The frequency range for TRFU is calculated for disruption based on target spindle size during the late prophase to telophase phases of mitosis. The average spindle size of mitotic neoplastic cells from the patient sample is characterized at 5 microns. TRFU is calculated at 0.5 to 3.0 times 5 microns, providing a frequency range of 150 MHz to 900 MHz to resonate along the spindle length.

The frequency range for TRFU is further calculated to provide a bandwidth that is discrete at either 0.5, 1.0, 2.0 or 3.0 times the frequency fundamental.

Treatment of the patient with TRFU is as follows. Each body location identified as containing neoplastic cells is exposed to three out of four of the following TRFU frequencies: 25 MHz, 50 MHz, 100 MHz, and 150 MHz. Power intensity is set at 100 to 10,000 W/m². Exposure to TRFU is for one hour per frequency and power level selected, once a week, for twelve weeks. Reduction in neoplastic cells is determined by diagnostic methods known in the art.

Following TRFU treatment, patient's cancer regresses.

What is claimed is:

1. A method for reducing or eliminating neoplastic cells in a subject comprising administering to said subject a therapeutically effective treatment regime of tuned resonant frequency ultrasound energy, wherein said resonant frequency is calculated to resonate with cellular structures of mitotic cells, and wherein said treatment regime reduces or eliminates said neoplastic cells.

2. The method of claim 1, wherein the tuned resonant frequency bandwidth is calculated to be 0.5 to 3.0 times the average cellular diameter or major axis dimension of a mitotic neoplastic cell or cellular structure of said neoplastic cell population, such cell being in the anaphase to telophase stage of mitosis.

3. The method of claim 2, wherein the tuned resonant frequency range is 25-150 MHz.

4. The method of claim 1, wherein the tuned resonant frequency bandwidth is calculated to be 0.5 to 3.0 times the average nuclear diameter of a mitotic neoplastic cell of said neoplastic cell population, such cell being in the telophase stage of mitosis.

5. The method of claim 4, wherein the tuned resonant frequency range is 62-375 MHz.

6. The method of claim 1, wherein the tuned resonant frequency bandwidth is calculated to disrupt the average spindle or DNA fragment length of a mitotic neoplastic cell of said neoplastic cell population, such cell being in the metaphase to telophase stage of mitosis.

7. The method of claim 6, wherein the tuned resonant frequency range is 250-1,500 MHz.

8. A method for reducing or eliminating neoplastic cells in a subject comprising administering to said subject a therapeutically effective treatment regime of tuned resonant frequency ultrasound energy, wherein said resonant frequency is within the range 1 MHz to 1,500 MHz, and wherein said treatment regime reduces or eliminates said neoplastic cells.

9. A method for reducing or eliminating neoplastic cells in a subject comprising administering to said subject a therapeutically effective treatment regime of tuned resonant frequency ultrasound energy, wherein said regime comprises administration of two or more different resonant frequencies and wherein the two or more different frequencies fall within two or more of the following ranges:
a. 0.5 to 3.0 times the average cellular diameter of a mitotic neoplastic cell of said neoplastic cell population, such cell being in the anaphase to telophase stage of mitosis;
b. 0.5 to 3.0 times the average nuclear diameter of a mitotic neoplastic cell of said neoplastic cell population, such cell being in the interphase to early prophase stages of mitosis;
c. 0.5 to 3.0 times the average spindle length of a mitotic neoplastic cell of said neoplastic cell population, such cell being in the late prophase to telophase stage of mitosis;
wherein said treatment regime reduces or eliminates said neoplastic cells.

10. A method for reducing or eliminating neoplastic cells in a subject comprising administering to said subject a therapeutically effective treatment regime of tuned resonant frequency ultrasound energy, wherein said regime comprises administration of two or more different resonant frequencies and wherein the two or more different frequencies fall within two or more of the following ranges:
a. 25-150 MHz;
b. 62-375 MHz;
c. 205-1,500 MHz;
wherein said treatment regime reduces or eliminates said neoplastic cells.

11. The method of claim 1, further comprising administering to said subject one or more additional anticancer treatments.

12. The method of claim 1, wherein the tuned resonant frequency bandwidth is continuous between 0.5 to 3.0 times the frequency fundamental.

13. The method of claim 1, wherein the passband of the ultrasonic energy is about 0.5, 1.0, 2.0 or 3.0 times the frequency fundamental.

14. The method of claim 13, wherein the passband of the ultrasonic energy further includes one or more of: 1.5, 2.5 or 3.5 times the frequency fundamental.

15. The method of claim 2, further comprising administering to said subject one or more additional anticancer treatments.

16. The method of claim 3, further comprising administering to said subject one or more additional anticancer treatments.

17. The method of claim 4, further comprising administering to said subject one or more additional anticancer treatments.

18. The method of claim 5, further comprising administering to said subject one or more additional anticancer treatments.

19. The method of claim 6, further comprising administering to said subject one or more additional anticancer treatments.

20. The method of claim 7, further comprising administering to said subject one or more additional anticancer treatments.

21. The method of claim 8, further comprising administering to said subject one or more additional anticancer treatments.

22. The method of claim 9, further comprising administering to said subject one or more additional anticancer treatments.

23. The method of claim 10, further comprising administering to said subject one or more additional anticancer treatments.

24. The method of claim 12, further comprising administering to said subject one or more additional anticancer treatments.
